(19) United States
(12) Patent Application Publication
(10) Pub. No.: US 2008/0119681 A1
(43) Pub. Date: May 22, 2008

(54) Process for the Production of Olefins by Autothermal Cracking

(75) Inventors: Andrew Lindsay Burns, Perthshire (GB); Ian Allan Beattie Reid, London (GB)

Correspondence Address: NIXON & VANDERHYE, PC, 901 NORTH GLEBE ROAD, 11TH FLOOR, ARLINGTON, VA, 22203, US

(73) Assignee: Ineos Europe Limited, Lyndhurst, Hampshire (GB)

(21) Appl. No.: 11/795,818

(22) PCT Filed: Dec. 22, 2005
(86) PCT No.: PCT/GB05/05048

§ 371 Date: Oct. 5, 2007

(30) Foreign Application Priority Data
Jan. 21, 2005 (GB) 0501255.4

(51) Int. Cl. C07C 4/04

(52) U.S. Cl. 585/653

(57) ABSTRACT

The present invention provides a process for the production of olefins by autothermal cracking of a liquid paraffinic hydrocarbon-containing feedstock in the presence of a molecular oxygen-containing gas, wherein said process comprises (a) providing a liquid paraffinic hydrocarbon-containing feedstock, (b) mixing said liquid paraffinic hydrocarbon-containing feedstock with a diluent comprising steam, said diluent being pre-heated to a temperature of at least 300° C., to produce a vaporised diluent liquid paraffinic hydrocarbon-containing feedstream comprising at least 20% by volume of steam, (c) subsequently mixing said vaporised diluted liquid paraffinic hydrocarbon-containing feedstream with a molecular oxygen-containing gas to produce a diluted mixed feedstream, (d) subsequently contacting said diluted mixed feedstream with a catalyst capable of supporting combustion beyond the normal fuel rich limit of flammability, to provide a hydrocarbon product stream comprising olefins.

The present invention relates to a process for the production of olefins. In particular, the present invention relates to a process for the production of olefins by autothermal cracking.

Autothermal cracking is a route to olefins in which the hydrocarbon feed is mixed with oxygen and passed over an autothermal cracking catalyst. The autothermal cracking catalyst is capable of supporting combustion beyond the fuel rich limit of flammability. Combustion is initiated on the catalyst surface and the heat required to raise the reactants to the process temperature and to carry out the endothermic cracking process is generated in situ. Generally the hydrocarbon feed and molecular oxygen is passed over a supported catalyst to produce the olefin product. Typically, the catalyst comprises at least one platinum group metal, for example, platinum. The autothermal cracking process is described in EP 332289B; EP-529793B; EP-A-0709446 and WO 00/14035.

It is known that additional feed components may also be passed to the autothermal cracker. Suitable additional feed components include, for example, hydrogen and steam. Hydrogen, for example, is typically fed because it reacts preferentially with the molecular oxygen-containing gas to generate the heat required for autothermal cracking of the hydrocarbon feed, reducing the requirement to burn the more valuable hydrocarbon feed to generate said heat.

We have now found that the autothermal cracking of liquid hydrocarbons may be advantageously operated by using a diluent comprising steam which is pre-mixed with the liquid hydrocarbon.

Hence, in a first aspect, the present invention provides a process for the production of olefins by autothermal cracking of a liquid paraffinic hydrocarbon-containing feedstock in the presence of a molecular oxygen-containing gas, wherein said process comprises
(a) providing a liquid paraffinic hydrocarbon-containing feedstock,
(b) mixing said liquid paraffinic hydrocarbon-containing feedstock with a diluent comprising steam, said diluent being pre-heated to a temperature of at least 300° C., to produce a vaporised diluted liquid paraffinic hydrocarbon-containing feedstream comprising at least 20% by volume of diluent,
(c) subsequently mixing said vaporised diluted liquid paraffinic hydrocarbon-containing feedstream with a molecular oxygen-containing gas to produce a diluted mixed feedstream,
(d) subsequently contacting said diluted mixed feedstream with a catalyst capable of supporting combustion beyond the normal fuel rich limit of flammability, to provide a hydrocarbon product stream comprising olefins.

“Liquid paraffinic hydrocarbon” as used herein refers to paraffinic hydrocarbons which are liquid at standard temperature and pressure (s.t.p.).

Suitable liquid hydrocarbons for the process of the present invention include naphtha, gas oils, vacuum gas oils and mixtures thereof.

Step (b) of the process of the present invention comprises mixing said liquid paraffinic hydrocarbon-containing feedstock with a diluent comprising steam, said diluent being pre-heated to a temperature of at least 300° C., to produce a vaporised diluted liquid paraffinic hydrocarbon-containing feedstream comprising at least 20% by volume of diluent.

Thus, step (b) comprises vaporisation of the liquid paraffinic hydrocarbon-containing feedstock. This may be achieved by vaporising the liquid paraffinic hydrocarbon-containing feedstock before mixing with the diluent, but preferably, the liquid paraffinic hydrocarbon-containing feedstock may be mixed with the diluent and simultaneously or subsequently vaporised. Preferably, the pre-heated diluent is used, at least in part, to vaporise the liquid paraffinic hydrocarbon-containing feedstock.

The use of the diluent before or during vaporisation of the feedstock or the addition of the diluent to the already vaporised feedstock reduces the risk of auto-ignition of the vaporised feedstock. In particular, vaporised liquid hydrocarbons generally have only a narrow temperature window between the low temperature and high temperature regions where auto-ignition can occur. This window is pressure dependent and reduces as pressure increases. Thus, it is desirable to have good (narrow range) temperature control and low residence time for the vaporised feedstock. In addition, it generally becomes harder to vaporise liquid hydrocarbons as the pressure increases, so although it is desirable to reduce the residence time of the vaporised liquid hydrocarbon as pressure increases this becomes difficult due to the difficulty of vaporising the liquid hydrocarbon in the first place. The use of a diluent according to the process of the present invention reduces the partial pressure of the vaporised hydrocarbon whilst keeping the overall pressure significantly higher. Thus, the stable temperature window for the vaporised hydrocarbon is larger than for the equivalent total pressure, and the residence time is less of an issue. The dilution of the mixed feedstream by the diluent also allows higher flow rates to be obtained which makes mixing of the liquid paraffinic hydrocarbon-containing stream with the molecular oxygen containing gas quicker and easier. (In general, mixing of the hydrocarbon-containing stream and the molecular oxygen containing gas is most efficient when flow rates of the molecular oxygen containing gas and the hydrocarbon containing stream are in the ratio 2:1 to 5:1. In the absence of other components, to obtain suitable molar ratios of hydrocarbon and oxygen in the present invention, the flow rates of liquid hydrocarbons required, even after vaporisation, are much lower than the flow rates of oxygen required, but the addition of diluent to the liquid hydrocarbon according to the present invention reduces this difference). In addition, the higher flow rates obtained allow feeding of the diluted mixed feedstream to the catalyst within a shorter residence time, again reducing ignition issues.

In addition, the diluent can be used to aid vaporisation of the liquid hydrocarbon.

In general, higher total pressures are desired because they can lead to improved selectivity. A lower partial pressure of liquid paraffinic hydrocarbon-containing feedstock will also lead to a reduced partial pressure of products in the product stream, which will reduce further reactions taking place in the product stream, and hence reduce the quench requirements for the product stream.

A heat exchanger may be employed to pre-heat the diluent prior to mixing. The diluent is preferably pre-heated to a temperature in the range 300° C. to 400° C.

In addition to steam, or in a further embodiment alternatively, the diluent may comprise carbon monoxide, carbon dioxide, an inert gas, such as helium, neon, argon or nitrogen, or a mixture thereof.

Carbon monoxide and carbon dioxide, for example, may be obtained as by-products from the autothermal cracking process of step (d).

A preferred diluent comprises 20 to 100% by volume of steam, more preferably 50 to 100% by volume of steam and most preferably at least 75% by volume of steam.

The vaporised diluted liquid paraffinic hydrocarbon-containing feedstream preferably comprises at least 20% by volume of steam, such as at least 40% by volume of steam.

Typically, the diluted mixed feedstream comprises 20 to 80% by volume of diluent, such as 40 to 60% by volume.

Most preferably, the diluted mixed feedstream comprises 20 to 80% by volume of steam, such as 40 to 60% by volume of steam.

The diluent may be mixed with the liquid paraffinic hydrocarbon-containing feedstock using any suitable mixing device.

A preferred method of introducing the diluent is by use of a sparger.

The diluent may be used to introduce quantities of other hydrocarbons (being hydrocarbons other than the liquid paraffinic hydrocarbon-containing feedstock) to the process of the present invention. Hence, the diluent may also comprise up to 20% by volume of hydrocarbons other than the liquid paraffinic hydrocarbon-containing feedstock, for example of dienes, such as butadiene and/or of hydrocarbons which are gases at room temperature and pressure.

The diluent may also be used to deliver quantities of hydrogen at high temperature to the reaction, and hence the diluent may comprise up to 20% by volume of hydrogen.

Alternatively, in the absence of hydrocarbons or hydrogen in the diluent, the diluent may comprise up to 20% by volume of molecular oxygen.

The addition of steam has the further advantage that steam will inhibit formation of pyrolytic carbon on the catalyst and the formation of acetylenes in the cracking reaction. Steam (water) is also easier to remove from the product stream than diluents which are gaseous at standard temperature and pressure, such as nitrogen, carbon monoxide and carbon dioxide. Typically, the steam (water) will be recovered as an aqueous phase during product stream treatment, for example in the product quench usually used to cool the reaction, and can therefore be easily separated from the product gases.

In one embodiment, the pre-heated diluent comprising steam may be produced by providing a stream comprising hydrogen and molecular oxygen, which react to produce steam (water) and generate the heat required to heat the stream to the required pre-heat temperature.

In an alternative embodiment, the pre-heated diluent comprising steam may be produced by providing a stream comprising methane (and optionally hydrogen) and reacting this with molecular oxygen, to produce a hot stream comprising steam (water), carbon dioxide and, optionally, any unreacted methane, at least some of which is used as the pre-heated diluent. The hot stream comprising steam produced from hydrogen and molecular oxygen or steam, carbon dioxide and any unreacted methane produced from methane and molecular oxygen is typically initially at a temperature of much higher than 400° C. and, hence, much higher than that required for the diluent stream. The stream may be cooled by heat exchange and/or diluted to produce the diluent stream of the desired temperature. Where the stream is cooled by heat exchange the heat removed may be used as pre-heat for other feeds to the process, such as the molecular oxygen-containing gas as described below.

Preferably, at least some of the steam used as diluent may be obtained from downstream processing steps, such as from the quench used to cool the reaction. A further suitable source of steam is process water, which as used herein is defined as water formed by reaction in the process of the invention.

Prior to recycle and use as steam, any water from the downstream processing steps may be treated in order that it may be fed to a boiler and vaporized without causing undue fouling. Suitable treatment steps may include removal of organic liquid components, removal of solids, and treatment to adjust the acidity of the water (to avoid corrosion issues). Components which will not cause undue fouling in the vaporization stage may be left in the stream and will be at least partially consumed in the reaction zone.

In step (c) of the present invention, the vaporised diluted liquid paraffinic hydrocarbon-containing feedstream is subsequently mixed with a molecular oxygen-containing gas to produce a diluted mixed feedstream.

Any suitable molecular oxygen-containing gas may be used. Suitably, the molecular oxygen-containing gas is molecular oxygen, air and/or mixtures thereof. The molecular oxygen-containing gas may be mixed with an inert gas such as nitrogen or argon.

The molecular oxygen-containing gas may be pre-heated prior to mixing. When pre-heated, the molecular oxygen-containing is typically pre-heated to less than 150° C., preferably less than 100° C.

Generally, the amount of pre-heating of the various streams that are mixed is limited to temperatures wherein the diluted, mixed feedstream will be below the autoignition temperature of the mixture. This is usually significantly below the reaction temperature obtained when the mixed feedstream contacts the catalyst. Typically, the diluted, mixed feedstream produced will be at a temperature in the range 250° C. to 500° C., such as in the range 350° C. to 450° C., although the preferred range will be pressure dependent.

Preferably the diluted mixed feedstream comprises paraffinic hydrocarbons (liquid paraffinic hydrocarbons and, optionally any other reactive paraffinic hydrocarbons that may be introduced) at a ratio of paraffinic hydrocarbon to molecular oxygen-containing gas of 5 to 16 times, preferably 5 to 13.5 times, more preferably 6 to 10 times, the stoichiometric ratio of paraffinic hydrocarbon to molecular oxygen-containing gas required for complete combustion of the hydrocarbon to carbon dioxide and water.

Hydrogen (molecular hydrogen) may be co-fed to the process of the present invention as a component of the diluted mixed feedstream. Suitably, the molar ratio of hydrogen to molecular oxygen-containing gas is in the range 0.2 to 4, preferably, in the range 1 to 3.

Preferably, hydrogen is pre-mixed with the liquid paraffinic hydrocarbon-containing feedstock before mixing with the molecular oxygen-containing gas.

The use of a hot diluent reduces the heating requirements of the diluted mixed feedstream compared to addition of a cold diluent. The use of a hot diluent also has advantages in the start-up and shut-down of the autothermal cracking reaction. During start-up, the hot diluent can be introduced to the catalyst before the reactants, causing the catalyst to be pre-heated to the temperature of the diluent. When the reactants are introduced the catalyst rapidly heats to reaction temperature, which is typically in the range 600° C. to 1200° C. at the exit of the catalyst. Because the catalyst is already at a higher temperature from use of hot diluent prior to introduction of the reactants, the thermal stresses across the catalyst on initiation of reaction are reduced.

Similarly, on shut-down, the thermal stresses across the catalyst can be reduced by using the hot diluent, optionally with a purge gas such as nitrogen, rather than the purge gas alone.

In step (d) of the present invention the diluted mixed feedstream is contacted with a catalyst capable of supporting combustion beyond the normal fuel rich limit of flammability, to provide a hydrocarbon product stream comprising olefins.

The catalyst capable of supporting combustion beyond the fuel rich limit of flammability usually comprises a Group VIII metal as its catalytic component. Suitable Group VIII metals include platinum, palladium, ruthenium, rhodium, osmium and iridium. Rhodium, and more particularly, platinum and palladium are preferred. Typical Group VIII metal loadings range from 0.01 to 100 wt %, preferably, between 0.01 to 20 wt %, and more preferably, from 0.01 to 10 wt % based on the total dry weight of the catalyst.

The reaction may suitably be carried out at a catalyst exit temperature in the range 600° C. to 1200° C., preferably, in the range 850° C. to 1050° C. and, most preferably, in the range 900° C. to 1000° C.

The process of the present invention is preferably operated at an elevated pressure of at least 1 barg (total pressure of diluted mixed feedstream), most preferably in the range 1 to 5 barg. The process of the present invention is preferably operated at a partial pressure of liquid paraffinic hydrocarbon-containing feedstock and molecular oxygen containing gas in the diluted mixed feedstream of greater than 0.5 barg, such as in the range 0.5 to 4 barg.

The diluted mixed feedstream is passed over the catalyst at a gas hourly space velocity which is pressure dependent and typically greater than 10,000 h⁻¹ barge⁻¹, preferably greater than 20,000 h⁻¹ barg⁻¹ and, most preferably, greater than 100,000 h⁻¹ barg⁻¹. For example, at 20 barg pressure, the gas hourly space velocity is most preferably, greater than 2,000,000 h⁻¹. It will be understood, however, that the optimum gas hourly space velocity will depend upon the nature of the feed composition.

The reaction products are preferably quenched with water as they emerge from the autothermal cracker, typically in a suitable quench tower.

To avoid further reactions taking place, usually the product stream is cooled to between 750-600° C. within 100 milliseconds of formation, preferably within 50 milliseconds of formation and most preferably within 20 milliseconds of formation. As noted previously, the use of a diluent according to the process of the present invention reduces the rate of further reactions taking place in the product stream compared to reactions in the absence of diluent. The present invention therefore provides the potential to eliminate the direct quench and replace it with more “conventional” heat recovery systems, such as a waste heat boiler.

The hydrocarbon product stream, in addition to olefins, may comprise unreacted paraffinic hydrocarbons, hydrogen, carbon monoxide, methane, and small amounts of acetylenes, aromatics and carbon dioxide, which need to be separated from the desired olefins.

Where a Group VII catalyst is employed, it is preferably employed in combination with a catalyst promoter. The promoter may be a Group IIIA, IVA, and/or VA metal. Alternatively, the promoter may be a transition metal; the transition metal promoter being a different metal to that which may be employed as the Group VIII transition metal catalytic component.

Preferred Group IIIA metals include Al, Ga, In and Tl. Of these, Ga and In are preferred. Preferred Group IVA metals include Ge, Sn and Pb. Of these, Ge and Sn are preferred. The preferred Group VA metal is Sb. The atomic ratio of Group VIII B metal to the Group IIIA, IVA or VA metal may be 1:0.1-50.0, preferably, 1:0.1-12.0.

Suitable metals in the transition metal series include those metals in Group IB to VII of the Periodic Table. In particular, transition metals selected from Groups IB, IIB, VIB, VIIB and VIII of the Periodic Table are preferred. Examples of such metals include Cr, Mo, W, Fe, Ru, Os, Co, Rh, Ir, Ni, Pt, Cu, Ag; Au, Zn, Cd and Hg. Preferred transition metal promoters are Mo, Rh, Ru, Ir, Pt, Cu and Zn. The atomic ratio of Group VIII metal to transition metal promoter may be 1:0.1-50.0, preferably, 1:0.1-12.0.

Preferably, the catalyst comprises only one promoter; the promoter being selected from Group IIIA, Group IVA, Group VB and the transition metal series. For example, the catalyst may comprise a metal selected from rhodium, platinum and palladium and a promoter selected from the group consisting of Ga, In, Sn, Ge, Ag, Au or Cu. Preferred examples of such catalysts include Pt/Ga, Pt/In, Pt/Sn, Pt/Ge, Pt/Cu, Pd/Sn, Pd/Ge, Pd/Cu and Rh/Sn. The Rh, Pt or Pd may comprise between 0.01 and 5.0 wt %, preferably, between 0.01 and 2.0 wt %, and more preferably, between 0.05 and 1.0 wt % of the total weight of the catalyst. The atomic ratio of Rh, Pt or Pd to the Group IIIA, IVA or transition metal promoter may be 1:0.1-50.0, preferably, 1:0.1-12.0. For example, atomic ratios of Rh, Pt or Pd to Sn may be 1:0.1 to 50, preferably, 1:0.1-12.0, more preferably, 1:0.2-3.0 and most preferably, 1:0.5-1.5. Atomic ratios of Pt or Pd to Ge, on the other hand, may be 1:0.1 to 50, preferably, 1:0.1-12.0, and more preferably, 1:0.5-8.0. Atomic ratios of Pt or Pd to Cu may be 1:0.1-3.0, preferably, 1:0.2-2.0, and more preferably, 1:0.5-1.5.

Alternatively, the promoter may comprise at least two metals selected from Group IIIA, Group IVA and the transition metal series. For example, where the catalyst comprises platinum, the platinum may be promoted with two metals from the transition metal series, for example, palladium and copper. Such Pt/Pd/Cu catalysts may comprise palladium in an amount of 0.01 to 5 wt %, preferably, 0.01 to 2 wt %, and more preferably, 0.01 to 1 wt % based on the total weight of the dry catalyst. The atomic ratio of Pt to Pd may be 1:0.1-10.0, preferably, 1:0.5-8.0, and more preferably, 1:1.0-5.0. The atomic ratio of platinum to copper is preferably 1:0.1-3.0, more preferably, 1:0.2-2.0, and most preferably, 1:0.5-1.5.

Where the catalyst comprises platinum, it may alternatively be promoted with one transition metal, and another metal selected from Group IIIA or Group IVA of the periodic table. In such catalysts, palladium may be present in an amount of 0.01 to 5 wt %, preferably, 0.01 to 2.0 wt %, and more preferably, 0.05-1.0 wt % based on the total weight of the catalyst. The atomic ratio of Pt to Pd may be 1:0.1-10.0, preferably, 1:0.5-8.0, and more preferably, 1:1.0-5.0. The atomic ratio of Pt to the Group IIIA or IVA metal may be 1:0.1-60, preferably, 1:0.1-50.0. Preferably, the Group IIIA or IVA metal is Sn or Ge, most preferably, Sn.

For the avoidance of doubt, the Group VIII metal and promoter in the catalyst may be present in any form, for example, as a metal, or in the form of a metal compound, such as an oxide.

The catalyst may be unsupported, such as in the form of a metal gauze, but is preferably supported. Any suitable support material may be used, such as ceramic or metal supports, but ceramic supports are generally preferred. Where ceramic supports are used, the composition of the ceramic support may be any oxide or combination of oxides that is stable at high temperatures of, for example, between 600° C. and 1200° C. The support material preferably has a low thermal expansion co-efficient, and is resistant to phase separation at high temperatures.

Suitable ceramic supports include corderite, lithium aluminium silicate (LAS), alumina (α-Al₂O₃), yttria stabilised zirconia, alumina titanate, niascon, and calcium zirconyl phosphate. The ceramic supports may be wash-coated, for example, with γ-Al₂O₃.

The support is preferably in the form of a foam or a honeycomb monolith.

The catalyst capable of supporting combustion beyond the fuel rich limit of flammability may be prepared by any method known in the art. For example, gel methods and wet-impregnation techniques may be employed. Typically, the support is impregnated with one or more solutions comprising the metals, dried and then calcined in air. The support may be impregnated in one or more steps. Preferably, multiple impregnation steps are employed. The support is preferably dried and calcined between each impregnation, and then subjected to a final calcination, preferably, in air. The calcined support may then be reduced, for example, by heat treatment in a hydrogen atmosphere.

1-8. (canceled)

9. A process for the production of olefins by autothermal cracking of a liquid paraffinic hydrocarbon-containing feedstock in the presence of a molecular oxygen-containing gas, wherein said process comprises
(a) providing a liquid paraffinic hydrocarbon-containing feedstock,
(b) mixing said liquid paraffinic hydrocarbon-containing feedstock with a diluent comprising steam, said diluent being pre-heated to a temperature of at least 300° C., to produce a vaporised diluted liquid paraffinic hydrocarbon-containing feedstream comprising at least 20% by volume of diluent,
(c) subsequently mixing said vaporised diluted liquid paraffinic hydrocarbon-containing feedstream with a molecular oxygen-containing gas to produce a diluted mixed feedstream,
(d) subsequently contacting said diluted mixed feedstream with a catalyst capable of supporting combustion beyond the normal fuel rich limit of flammability, to provide a hydrocarbon product stream comprising olefins.

10. A process as claimed in claim 9 wherein the pre-heated diluent is used, at least in part, to vaporise the liquid paraffinic hydrocarbon-containing feedstock.

11. A process as claimed in claim 9 wherein the liquid paraffinic hydrocarbon-containing feedstock comprises naphtha, gas oils, vacuum gas oils or a mixture thereof.

12. A process as claimed in claim 9, wherein the diluted mixed feedstream comprises paraffinic hydrocarbons at a ratio of paraffinic hydrocarbon to molecular oxygen-containing gas of 5 to 16 times, preferably 5 to 13.5 times, more preferably 6 to 10 times, the stoichiometric ratio of paraffinic hydrocarbon to molecular oxygen-containing gas required for complete combustion of the hydrocarbon to carbon dioxide and water.

13. A process as claimed in claim 9, wherein the diluted mixed feedstream comprises 20 to 80% by volume of steam, such as 40 to 60% by volume.

14. A process as claimed in claim 9, wherein the diluent comprises 50 to 100% by volume of steam.

15. A process as claimed in claim 9, wherein the diluent also comprises up to 20% by volume of hydrocarbons other than methane or the liquid paraffinic hydrocarbon-containing feedstock.

16. A process as claimed in claim 9, wherein the catalyst capable of supporting combustion beyond the fuel rich limit of flammability usually comprises a Group VIII metal as its catalytic component.
